(19) United States
(12) Patent Application Publication
(10) Pub. No.: US 2025/0235682 A1
(43) Pub. Date: Jul. 24, 2025

(54) SURGICAL EVACUATION PORT DEVICE WITH SEALING DEVICE

(71) Applicant: Asfora IP, LLC, Sioux Falls, SD (US)

(72) Inventor: Wilson Theophilo Asfora, Sioux Falls, SD (US)

(21) Appl. No.: 18/985,937

(22) Filed: Dec. 18, 2024

Related U.S. Application Data
(60) Provisional application No. 63/563,087, filed on Mar. 8, 2024.
(60) Provisional application No. 63/611,728, filed on Dec. 18, 2023.

(51) Int. Cl. A61M 39/02; A61M 1/00

(52) U.S. Cl. CPC A61M 39/0247; A61M 1/772; A61M 2039/025; A61M 2039/0264; A61M 2039/027; A61M 2039/0279; A61M 2039/0291; A61M 2210/0693

(57) ABSTRACT

A surgical evacuation port device for removing fluid from a surgical site including a body having a distal opening, a primary evacuation opening, and a primary lumen extending from the distal opening to the primary evacuation opening. The surgical evacuation port device also includes an access port defining an access port lumen that is in fluid communication with the primary lumen. A sealing device and/or valve is coupled to the access port and configured to receive an access tool while maintaining pressure within the primary lumen.

### CROSS-REFERENCE TO RELATED APPLICATIONS

This application claims under 35 U.S.C. § 119 (e) the benefit of U.S. Provisional Application No. 63/611,728, filed Dec. 18, 2023, and U.S. Provisional Application No. 63/563,087, filed on Mar. 8, 2024, the entire contents of which are incorporated by reference herein.

### BACKGROUND

### (a) Technical Field

The present disclosure relates to systems and methods for removing fluids from the subdural region of a patient, more particularly, to a surgical evacuation port device with an access tool allowing access to a surgical evacuation device via a blunt or sharp access tool.

### (b) Description of the Related Art

The subdural space of the human head is the space located between the brain and the lining of the brain, which is referred to as the dura mater (hereinafter referred to as the “dura”). Hemorrhages on the surface of the brain, for example, may cause a condition known as a subdural hematoma. The subdural hemorrhages may have a number of causes. For example, elderly persons may be more susceptible to subdural hemorrhages because as the brain ages it tends to become atrophic and the subdural space between the brain and the dura gradually enlarges. Bridging veins between brain and dura frequently stretch and rupture as a consequence of relatively minor head injuries, thus giving rise to a collection of blood in the subdural space. Further, severe linear acceleration or deceleration of the brain can result in the brain moving excessively with respect to the dura, often causing rupture of the bridging veins or the blood vessels on the surface of the brain, which can in turn cause subdural hemorrhages in an otherwise healthy brain.

These subdural blood collections can be classified as acute subdural hematomas, subacute subdural hematomas, and chronic subdural hematomas. Acute subdural hematomas, which are associated with major cerebral trauma, generally consist primarily of fresh blood. Subacute subdural hematomas are generally associated with less severe injuries than those underlying the acute subdural hematomas. Chronic subdural hematomas are generally associated with even less severe, or relatively minor, injuries. The chronic subdural hematomas tend to be less dense liquid consisting of very diluted blood. Another condition involving a subdural collection of fluid is a hygroma, which is a collection of cerebrospinal fluid (sometimes mixed with blood) beneath the dura, which may be encapsulated.

Devices such as subdural evacuation port devices are generally known and are capable of draining fluid from the subdural region. In some cases, blockages occur within the subdural evacuation port device, and it is desirable to remove the blockage. In some cases, it is useful to insert a blunt or sharp access tool.

The present disclosure describes systems and methods related to the above.

### SUMMARY

In one aspect of the present disclosure, a surgical evacuation port device includes a body that includes a distal opening, a primary evacuation opening, and a primary lumen extending from the distal opening to the primary evacuation opening. The device also includes an access port defining an access port lumen that is in fluid communication with the primary lumen and a sealing device mounted at a proximal end of the access port and configured to receive an access tool while maintaining pressure within the primary lumen. The sealing device includes a housing configured for mounting the sealing device at the proximal end of the access port and an inner tubular element having an outer surface. The outer surface of the inner tubular element is configured to form a seal with the access port. The sealing device further includes a sealing device lumen defined by the inner tubular element and disposed within the housing

In another aspect of the present disclosure, the inner tubular element is arranged concentrically within the housing.

In another aspect of the present disclosure, the outer surface of the inner tubular element forms an interference fit with an inner surface of the access port, the interference fit configured to form the seal with the access port.

In another aspect of the present disclosure, the outer surface of the inner tubular element is wider at a proximal end than at a distal end to form a taper, the distal end configured to fit within the access port.

In another aspect of the present disclosure, wherein the inner tubular element is configured for guiding the access tool through the sealing device.

In another aspect of the present disclosure, the inner tubular element extends distally from an inner wall of the housing and defines a lumen for receiving the access tool.

In another aspect of the present disclosure, the sealing device further comprises a resilient member configured to be penetrated by the access tool.

In another aspect of the present disclosure, the resilient member is arranged proximally from the inner tubular element.

In another aspect of the present disclosure, the resilient member is configured as a. resilient body having a passageway that extends longitudinally through the resilient body.

In another aspect of the present disclosure, the resilient member is made from a self-sealing material configured to seal punctures, cracks, or damage.

In another aspect of the present disclosure, the housing includes at least one coupling element on an inside wall configured to engage with a corresponding coupling element on the access port.

In another aspect of the present disclosure, the body is made from a rigid material with a substantially low thrombogenicity.

In another aspect of the present disclosure, the device is optically transparent.

In another aspect of the present disclosure, a valve includes a resilient body having a passageway that extends longitudinally through the resilient body, the valve being coupled to the sealing device.

In another aspect of the present disclosure, a surgical evacuation system includes the above device and a suction device configured to provide a suction force through the primary lumen, where the access tool includes at least one syringe.

In another aspect of the present disclosure, the syringe is operable with the surgical evacuation port device to cause negative pressure to assist with removing blockages or cause positive pressure for injecting medication.

In another aspect of the present disclosure, a surgical evacuation port device includes a body that includes a distal opening, a primary evacuation opening, and a primary lumen extending from the distal opening to the primary evacuation opening. The device also includes an access port defining an access port lumen that is in fluid communication with the primary lumen and a valve coupled to the access port and configured to receive an access tool while maintaining pressure within the primary lumen. The valve includes a resilient body having a passageway that extends longitudinally through the resilient body.

In another aspect of the present disclosure, the passageway forms a split septum provided at a proximal end of the resilient body.

In another aspect of the present disclosure, the resilient body includes at least one mating element configured to interface with at least one complimentary mating element within the access port lumen.

In another aspect of the present disclosure, the resilient body is made from a self-sealing material configured to seal punctures, cracks, or damage.

In another aspect of the present disclosure, the access port comprises at least one access port coupling configured to receive a corresponding mating portion on the access tool, thereby maintaining the access tool in a first position and at a first orientation.

In another aspect of the present disclosure, the access tool is a syringe.

In another aspect of the present disclosure, the syringe comprises a hollow needle.

In another aspect of the present disclosure, the body is made from a rigid material with a substantially low thrombogenicity.

In another aspect of the present disclosure, the device is optically transparent.

In another aspect of the present disclosure, a surgical evacuation system includes the above device and a suction device configured to provide a suction force through the primary lumen, where the access tool includes at least one syringe.

In another aspect of the present disclosure, the syringe is operable with the surgical evacuation port device to cause negative pressure to assist with removing blockages or cause positive pressure for injecting medication.

In another aspect of the present disclosure, a method for removing subdural fluids includes drilling a hole through a skull and a dura of a patient and mounting a surgical evacuation port device to the hole. The surgical evacuation port device includes a body that includes a distal opening, a primary evacuation opening, and a primary lumen extending from the distal opening to the primary evacuation opening. The surgical evacuation port device further includes an access port defining an access port lumen that is in fluid communication with the primary lumen and a valve coupled to the access port and configured to be penetrated by an access tool while maintaining pressure within the primary lumen. The sealing device includes a housing configured for mounting the sealing device at the proximal end of the access port and an inner tubular element having an outer surface. The outer surface of the inner tubular element is configured to form a seal with the access port. The sealing device further includes a sealing device lumen defined by the inner tubular element and disposed within the housing.

The method for removing subdural fluids also includes using a suction device configured to provide a suction force through a primary lumen of the surgical evacuation port device, evacuating subdural fluid from the dura through the primary lumen, inserting the access tool through the access port, and assisting with withdrawal or cleaning when access is required while the suction force is maintained in the primary lumen.

In another aspect of the present disclosure, the method further includes inserting a hollow needle through the access port lumen into the primary lumen.

In another aspect of the present disclosure, the method further includes injecting a thrombolytic agent via the hollow needle.

In another aspect of the present disclosure, the method further includes suctioning a blockage via the hollow needle.

In another aspect of the present disclosure, the method further includes irrigating the subdural space by administering sterile fluid through a first access port and evacuating said fluid through a second access port.

### BRIEF DESCRIPTION OF THE DRAWINGS

The following drawings are illustrative of particular embodiments of the present disclosure and therefore do not limit the scope of the present disclosure. Embodiments of the present disclosure will hereinafter be described in conjunction with the appended drawings, wherein like numerals denote like elements.

The inventive concepts are described with reference to the attached figures, wherein like reference numerals represent like parts and assemblies throughout the several views. Several aspects of the inventive concepts are described below with reference to example applications for illustration. It should be understood that numerous specific details, relationships, and methods are set forth to provide a full understanding of the inventive concepts. One having ordinary skill in the relevant art, however, will readily recognize that the inventive concepts can be practiced without one or more of the specific details or with other methods. In other instances, well-known structures or operation are not shown in detail to avoid obscuring the inventive concepts.

FIG. 1 shows an embodiment of a surgical evacuation system;

FIG. 2 is a side view of an example surgical evacuation port device of the surgical evacuation system of FIG. 1;

FIG. 3 is a perspective view of the surgical evacuation port device of FIG. 2;

FIG. 4 is a side view of another example surgical evacuation port device of the surgical evacuation system of FIG. 1; and

FIG. 5 is an enlarged perspective view of the surgical evacuation port device of FIG. 4.

### DETAILED DESCRIPTION

As discussed above, systems and devices for clearing the subdural space of the human brain are known (see, e.g., U.S. Pat. No. 7,694,821). Such systems generally include a subdural evacuation port device, and at least a device for evacuating a fluid from a subdural space of a patient via the subdural evacuation port device. Blockages can occur during evacuation of fluid from the subdural space which cause issues during evacuation. Blockages are removable from the subdural evacuation port device via known methods. For example, U.S. Patent Application Publication No. US 2024/0009439 describes methods of removing blockages using a tool inserted via an access port.

In some instances, it can be beneficial to utilize a subdural evacuation port for irrigating a subdural space, injecting medications, removing blockages, or perform other operations within the subdural space that may assist during a surgical procedure and/or with the patient's recovery or operation. During such operations or access to the subdural space (via a subdural evacuation port), it is important maintain an uninterrupted evacuation of fluid from the subdural space and/or a desired section pressure within the subdural evacuation port. The present disclosure relates to systems and methods for accessing a surgical evacuation port device using an access tool that accepts a variety of medical instruments such as sharp or blunt tools without interrupting the evacuation of fluid from the subdural space and/or while maintaining a desired suction pressure within the subdural evacuation port.

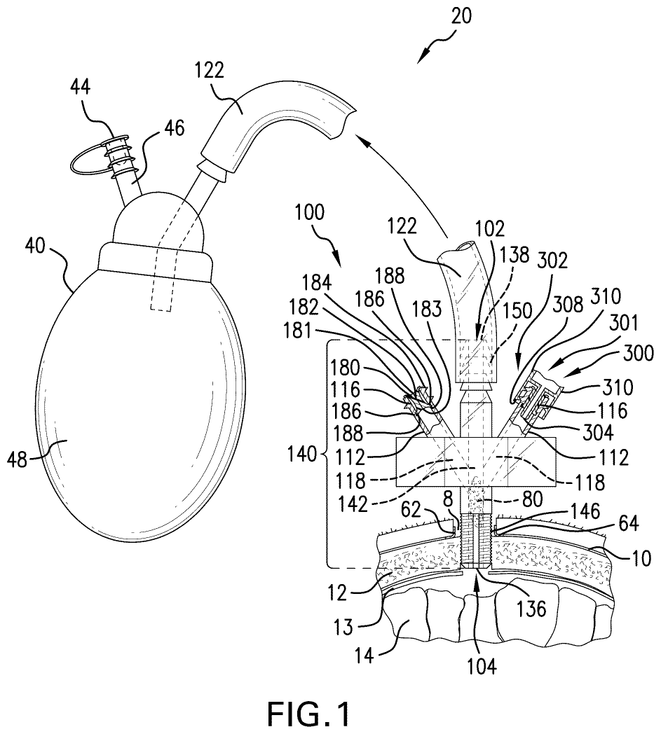

Referring to FIG. 1, a surgical evacuation system 20 in accordance with the principles of the present disclosure is shown. The surgical evacuation system 20 includes a surgical evacuation port device 100 and a suction device 40 that is configured to provide a suction force through a primary lumen 142 of the surgical evacuation port device 100. The suction force preferably is a negative pressure, a partial vacuum, aspiration, or any other force that can cause aspiration of the fluid from the subdural space without causing damage to and/or suction of tissue in and around the subdural space. The surgical evacuation port device 100 can be removably coupled to the suction device 40. The surgical evacuation system 20 additionally includes an access system 300 configured to provide access to a primary lumen 142 of the surgical evacuation port device 100 without interrupting the suction force from the suction device 40 and/or evacuation of fluid from the subdural space.

The surgical evacuation system 20 is depicted with the surgical evacuation port device 100 mounted at a hole 8 in a skull 12 (also known as a trephination) of a patient such that at least a portion of the surgical evacuation port device 100 extends through a dura 13 and into the subdural space above the brain 14 of the patient. In embodiments where fluid is drained through a hole, such as the hole 8, a drill device (not shown) is provided for drilling the hole into the bone of the patient after an incision has been made in a scalp 10 (e.g., subcutaneous tissue, the galea, and the periosteum) of the patient. A drill bit is mountable on the drill device in a suitable manner, such as by an adjustable chuck assembly, internally within the drill body or any other suitable arrangement. The drill bit can be rotated by a manual operation (e.g., turned by the surgeon's hand), by a motorized device, or any other suitable arrangement. Preferably, the drill device includes a drill stop for selectively limiting the maximum penetration of a tip of the drill into the skull 12 of the patient. The drill device can additionally be used in embodiments of the present disclosure.

In embodiments where the surgical evacuation port device 100 is used to drain fluid adjacent to a bone, a drill is not typically used, and an incision is made through scalp 10 of the patient. It will be appreciated that the surgical evacuation port device 100 can be configured to be mounted on and engage with any bone other than the skull 12 such as, without limitation, spine, hip, etc. for performing one or more of the functions disclosed herein. The surgical evacuation system 20 can additionally be configured to drain fluid near or adjacent to any other bone. In some embodiments, the drill may also be configured to pierce the dura 13 allowing fluid communication to the subdural space above the brain 14 in order to drain fluid therefrom. In other alternate embodiments, a retractor 62, 64 such as a tissue retractor, may be provided to hold the tissue (e.g., the scalp 10) apart to allow for the surgical evacuation port device 100 to be mounted within the hole 8. The retractor 62, 64 can be a Weitlaner retractor, Holzheimer retractor, or any other suitable retractor. In other embodiments, no retractor is provided, and the tissue is held apart by the surgical evacuation port device 100 itself.

The surgical evacuation port device 100 includes a bone engagement portion 146. The bone engagement portion 146 is configured to mount the port body 140 through the hole 8 in a bone to aspirate fluid from the bone and/or surrounding areas thereof, through the primary lumen 142 when a suction pressure is applied. The bone engagement portion 146 can additionally be configured to mount the port body 140 near any area that requires drainage of fluid therefrom. FIGS. 1-3 illustrate the bone engagement portion 146 being disposed around the distal end 136 of the surgical evacuation port device 100. The bone engagement portion 146 can, however, be positioned at any other suitable location allowing for engagement of the port body 140 with the bone. For example, the bone engagement portion 146 could be positioned near the center of port body 140 and extend downwardly as a flange with screws or be configured at any other suitable location to allow the device to engage with the bone (e.g., the skull 12).

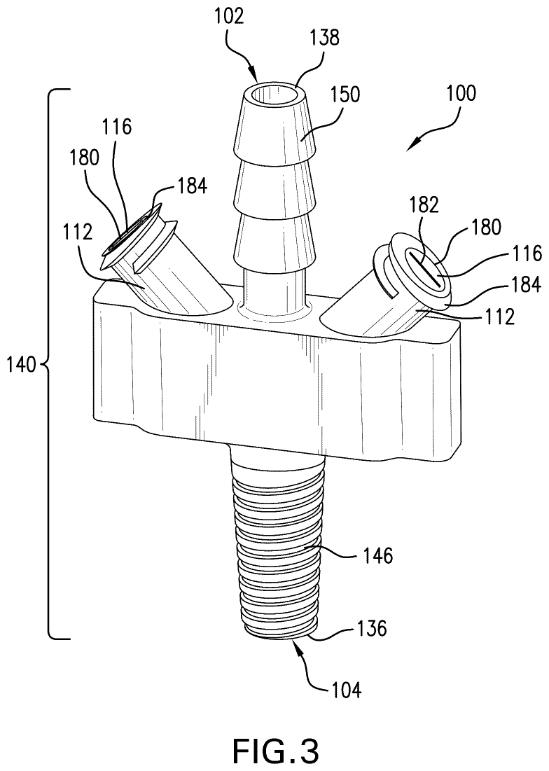

As shown in FIGS. 1-3, the bone engagement portion 146 includes threads. In some examples the threads include a flute (not shown) and are self-tapping. The bone engagement portion 146 can include any suitable type of threads. In other alternate embodiments, other engagement mechanisms allowing for engagement with a bone (e.g., the skull 12) are additionally possible such as, without limitation, friction fit, drilling, tapping, a flange having screws disposed thereon or any other suitable engagement mechanism. FIG. 3 shows an example embodiment of the bone engagement portion 146 with a pipe fitting. The pipe fitting includes pipe threads, which can be of various types, for example, male pipe threads [MPT], female pipe threads [FPT], national pipe threads [NPT] or Whitworth threads. Pipe threads can be configured such that the bone engagement portion 146 is tapered toward the distal end 136, while the primary lumen 142 remains substantially cylindrical.

In some embodiments, the bone engagement portion 146 includes threads that have a diameter of 4.5 mm, 5 mm, about 5.5 mm, about 6 mm about 7 mm, 7.5 mm, about 8, or any other suitable diameter. In some embodiments, the threads have a diameter between about 7-8 mm, about 7.2-7.8 mm, about 7.4-7.6 mm, about 4-6 mm, about 4.5 mm-5.5 mm, about 4.7-5.2 mm, or any other suitable range of diameters. In some other embodiments, the threads are configured as straight threads. In some other embodiments, the threads are trapezoidal threads. In some embodiments, the threads are pipe threads. In some examples, the threads are coarse and have a pitch of about 1 mm, about 1.5 mm, about 2 mm, about 2.5 mm or any other suitable pitch. In some examples, the threads have a pitch between about 1 mm-2.5 mm, about 1.2 mm-2.2 mm, about 1.4 mm-2 mm, or any other suitable range.

In various embodiments, the bone engagement portion 146 has an outside diameter that reduces or tapers from a proximal end towards a distal end thereof). The reduction in diameter may be uniform or non-uniform, stepped, etc. from the proximal end to the distal end. Such distally tapering diameter allows for better fixation of the device within the hole 8 formed in the skull 12 because the hole diameter can be configured such that narrower distal end can easily the hole while the broader proximal end forms a tight seal with the walls of the hole. Additionally, the diameter of the hole 8 can be selected to prevent insertion of the bone engagement portion 146 beyond a desired depth by only allowing insertion up to a certain diameter of the distally tapering diameter also prevents. In some example embodiments, the diameter of the bone engagement portion 146 at or near the distal end 136 is about 5-7 mm, about 5.5-6.5 mm, about 5 mm, about 6 mm, about 7 mm, or the like, and the diameter of the bone engagement portion 146 at or near its proximal end is about 7-9 mm, about 7.5-8.5 mm, about 7 mm, about 8 mm, about 9 mm, or the like. In some embodiments, the bone engagement portion 146 is a skull engagement portion and configured to be sized to fit within the hole 8 in the skull 12.

As shown in FIG. 1, the suction device 40 is coupled to the surgical evacuation port device 100. As discussed above, the suction device creates a suction force (uniform or non-uniform) through a primary lumen 142 of the surgical evacuation port device 100 and in a surgical area from which fluid needs to be evacuated (e.g., a subdural space) of the patient to allow built up fluid (or other materials) to be evacuated from the subdural space via the primary lumen 142.

The disclosure is not so limiting, and in various examples, the surgical evacuation system 20 can be used to evacuate fluid adjacent to a bone, within a different bone (e.g., not the skull 12) or any other suitable area that requires draining, fluid aspiration, irrigation, and/or suction therethrough. In some embodiments, the suction device 40 is coupled to the surgical evacuation port device 100 via a conduit 122, the conduit 122 being configured to removably attach to a fitting 150 on the surgical evacuation port device 100, in order to provide a uniform suction through the primary lumen 142 of the surgical evacuation port device 100. The fitting 150 surrounds a proximal end 138 of the port body 140 of the surgical evacuation port device 100 such that the conduit can be attached at the primary evacuation opening (e.g., the fitting 150 is discussed in greater detail below). In alternate embodiments, the suction device 40 can be directly attached to the proximal evacuation opening 102.

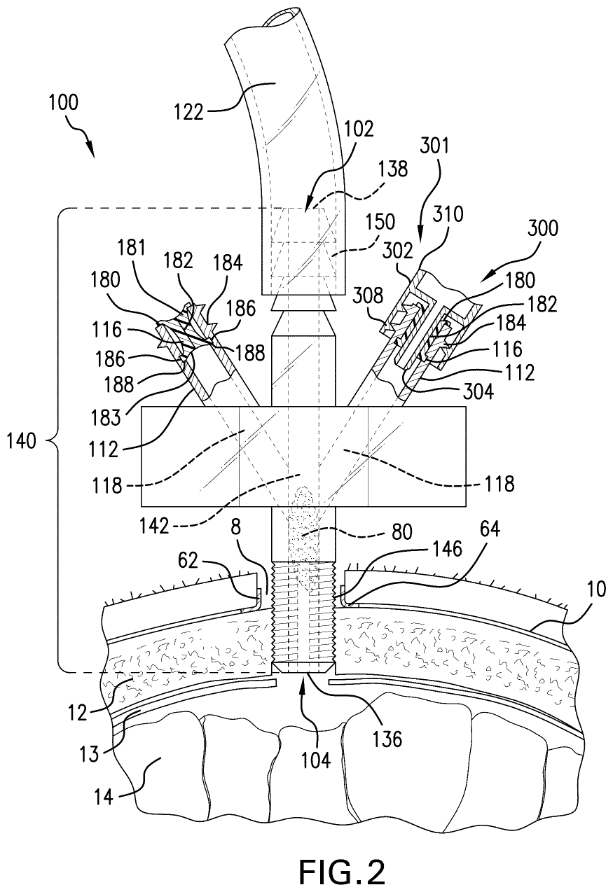

The surgical evacuation port device 100 is connected to a first end of the conduit 122 via the fitting 150. In FIGS. 1 and 2, e.g., the fitting 150 is depicted as a barb type fitting. However, the disclosure is not so limiting, and the fitting 150 may be any interface that connects the primary lumen 142 to the conduit 122 in an air-tight manner, including but not limited to a threaded fitting, a snap-fitting, or any other suitable fitting. A second end of the conduit 122 is attached, as discussed above, to the suction device 40.

The suction device 40 exerts a suction pressure (e.g., a negative pressure) for imparting a uniform partial vacuum (or suction) in the primary lumen 142 of the surgical evacuation port device 100. In some embodiments, the suction device 40 is a manual device such as a Jackson-Pratt bulb, or other suitable pump. In other embodiments, the suction device 40 is electronic or powered. The magnitude of the suction created is relatively low and exerts a gentle aspiration or suction in the subdural space when the suction device 40 is used to drain the subdural region. Substantial uniformity of the suction pressure is considered important for promoting the gradual re-expansion of the brain in the subdural space.

As depicted in FIG. 1, the suction device 40 is a manual suction device and includes a bulb pump. In the depicted embodiment, a conduit 122 (e.g., a hollow tube or pipe) connects the fitting 150 and the suction device 40 with one another. The suction device 40 includes a vent 46 for allowing air to escape. In certain examples, the vent 46 is a valve for expelling air from the suction device 40 when it is pumped and gently suctioning through the conduit 122 when air returns to the suction device 40. In some embodiments, the vent is a one-way valve, a ball-valve, or any other suitable valve or vent. In other alternate embodiments, the conduit 122 or the suction device 40 can include a vent or valve. The vent 46 or valve on the conduit 122 allows negative pressure to build within the surgical evacuation port device 100 while preventing positive pressure to enter the surgical evacuation port device 100.

The suction device 40 can additionally include a plug 44 instead of a one-way valve which can be inserted into vent 46. The plug can be inserted into the vent 46 after it has been pumped such that negative pressure is applied through the conduit. In certain examples, squeezing of suction device 40 can create a partial vacuum through the primary lumen 142 and gently suction out or aspirate fluid from the space from which fluid needs to be aspirated (e.g., a bone and surrounding area) into the primary lumen 142 and subsequently into the conduit 122. The conduit 122 can be formed from a material that is sufficiently rigid to not collapse under the suction applied by suction device 40. In certain embodiments, the conduit 122 can be formed from a transparent material. As shown in FIG. 1, the suction device 40 can include a reservoir 48 to contain or hold fluid suctioned by the surgical evacuation port device 100. In certain embodiments, the reservoir has a volume of about 100 ml, about 125 ml, about 150 ml, about 175 ml or any other suitable. In some other examples, the reservoir 205 has a volume of about 100 ml-175 ml, about 120 ml-160 ml, about 130 ml-150 ml, about 135 ml-145 ml, or any other suitable range of volumes.

In various embodiments, the magnitude of the suction pressure exerted by a typical evacuation device is about 0.8 inch to 1.0 inch of mercury (Hg) with respect to atmospheric pressure. Depending on the procedure, it will be appreciated that a lower level (e.g., less than 0.8 inches of mercury) of suction pressure may be used. While relatively higher levels of suction pressure may be used (such as up to approximately 1.2 inches of mercury), significantly higher levels of suction pressure can damage brain tissue and/or hamper the recovery of the brain and the associated tissues, by, for example, causing hemorrhages to occur.

An optimal suction pressure may be used that permits the suction pressure condition to be uniformly maintained in the subdural space of the patient for a relatively extended period of time for removing any further collection of fluid, as well as promoting a gradual expansion of the brain in the subdural space during the healing process. The components used to construct surgical evacuation port device 100 are sufficiently strong to sustain a suction of this level without collapsing (discussed in further detail below). As discussed above, it will be appreciated that any suitable evacuation device providing such pressure can be configured to attach to the fitting 150. In some examples, the negative pressure is about 0.5 in of Hg about 0.6 in of Hg, about 0.7 in of Hg, about 0.8 in/Hg, about 0.9 in of Hg, about 1.0 in of Hg, or any other suitable negative pressure. In some embodiments, the negative pressure is between about 0.5 in of Hg-1.0 in of Hg, about 0.55 in of Hg-0.95 in of Hg, about 0.7 in of Hg-0.9 in of Hg, about 0.75 in of Hg-0.85 in of Hg or any other suitable ranges of pressure.

As discussed above, a blockage 80 (e.g., due to a blood clot, thrombus, tissue debris, etc.) may occur in the primary lumen 142 and/or an area surrounding the distal opening of the primary lumen 142 during suction which prevents or slows the suction of fluid from the surgical site of interest. Furthermore, the blockage 80 can cause increased or nonuniform pressure within the surgical evacuation port device 100 and/or the surgical site, potentially causing or increasing the risk of tissue damage and hampering tissue recovery at the surgical site. Referring to FIGS. 1, 2, and 4, it will be appreciated that although the blockage 80 is shown in the primary lumen 142 of the surgical evacuation port device 100 as an agglomeration, the blockage 80 can take alternate forms, for example, a plurality of blockages.

If the blockage 80 is not detected and/or its cause or location is not timely or accurately determined, a user may deliberately increase the suction pressure through the primary lumen 142 if fluid suctioning is interrupted or slows. While this can assist with removing the blockage 80, the increased suction pressure can cause damage to the brain after the blockage 80 is cleared, particularly if the suction pressure is not immediately lowered once the blockage 80 is cleared. If the presence of the blockage 80 is not detected, then other unintended consequences may occur. For example, the user may cease the subdural evaluation procedure before all of the fluid in the subdural area is drained if fluid suction stops and/or slows due to the erroneous assumption that all fluid has been withdrawn or apply excessive suction pressure to the brain of the patient or apply a non-uniform suction through the primary lumen 142, or a host of other consequences. As such, if the blockage 80 occurs during aspiration of fluid from the surgical site of interest, the preferred response is to dissolve, reposition, and/or remove the blockage 80.

In U.S. patent application Ser. No. 18/347,515, filed on Jul. 5, 2023, which is incorporated by reference herein, a surgical evacuation port device that allows for a user or medical professional to optically detect and/or assess the location, type, size, or other properties of a blockage formed within the evacuation lumen (and/or the surgical site proximate the evacuation lumen) without removal of the surgical evacuation port device from the surgical site and/or without stopping the suctioning operation of the surgical evacuation port device (and, optionally, without the use of non-optical imaging technology). Additionally, the user is able to determine the methods for removal of the detected blockage (e.g., based on the location and properties such as size, density, etc.) as well as monitor effectiveness of a method for removal of the blockage (and adjust it for optimal removal).

U.S. Patent Application Publication No. US 2024/0009439, which is incorporated by reference herein, describes subdural evacuation port devices that include access ports for introducing access tools into the device primary lumen where the tool can perform an operation to assist in removal of the blockage. Examples of the operation can include, without limitation, delivery of various materials (e.g., thrombolytic agents) to dissolve or reduce the size of blockages, repositioning of the blockage, breaking up the blockage and/or suction or aspiration of the blockages through the tools. Needle access ports include an access port lumen in fluid communication with the evacuation lumen. The access port lumen and needle access ports are disposed and oriented within the subdural evacuation port and with respect to each other so as to allow a tool (e.g., a needle) to be inserted into the access port lumen and extend into the primary lumen and perform an operation to assist in removal of the blockage. For example, the needle can be used to withdraw a blockage from the primary lumen through the needle. Alternatively, tools can be used to deliver various agents (e.g., thrombolytic agents) at or near the blockage to dissolve or partially dissolve the blockage. For example, thrombolytics are medications used to dissolve blood clots, specifically thrombi by activating the fibrinolytic system. Some examples of thrombolytics that can be used include, but are not limited to: Alteplase, Reteplase, Tenecteplase, streptokinase or any other suitable, tissue plasminogen activator (tPA), streptokinase (SK), and urokinase (UK), or any other suitable thrombolytic agent. In other examples, a user can break up and/or reposition the blockage using other mechanisms. The present disclosure allows for a wider range of operations to be performed via the access ports and/or for a wider range of access tools than the above disclosures.

Referring to FIGS. 1-3, the surgical evacuation port device 100 includes a port body 140 having a distal end 136 and a proximal end 138. The distal end 136 includes a distal evacuation opening 104 and the proximal end 138 includes a proximal evacuation opening 102. In addition to the shape shown in FIGS. 1-3, the port body 140 can additionally be any other suitable shape such as substantially oval or rectangular. The primary lumen 142 extends through the port body 140 of the surgical evacuation port device 100 between the distal evacuation opening 104 and the proximal evacuation opening 102. The inner diameter of the primary lumen 142 is about 3.5-4.5 mm, about 3.6-4.4 mm, about 3.7-4.3 mm, about 3.8-4.2 mm, about 3.9-4.1 mm, about 3.8 mm, about 3.9 mm, about 4 mm, about 4.1 mm, about 4.2 mm, or the like. The length of the port body 140 between the proximal end 138 and the distal end 136 is about 4.5-5.5 cm, about 4.6-5.4 cm, about 4.7-5.3 cm, about 4.8-5.2 cm, about 4.9-5.1 cm, about 4.8 cm, about 4.9 cm, about 5 cm, about 5.1 cm, about 5.2 cm, or the like.

The port body 140 includes at least one access port 112. The access port 112 defines an access port lumen 118 that is in fluid communication with the primary lumen 142. The access port lumen 118 is disposed and oriented with respect to the port body 140 to allow an access tool 302 that is inserted into the access port lumen 118 to extend into the primary lumen 142. Alternatively, the access tool 302 may be inserted into the access port lumen 118 without reaching the primary lumen 142, which for example, is a useful arrangement for injecting a fluid into the primary lumen 142 via the access port lumen 118. In other alternate embodiments, access may be provided to the primary lumen 142 without interrupting suction of fluid from the subdural space, via the primary lumen 142.

In the embodiment shown in FIGS. 1-3, there are two access ports 112 disposed on opposite sides of the port body 140. In some examples, the access ports 112 can be configured as protrusions that extend away from the port body 140. The protrusions (not shown but discussed in the above referenced co-pending U.S. patent application Ser. No. 18/347,474) can be rigid, semi-rigid, or non-rigid protrusions. It will be appreciated that the surgical evacuation port device 100 can also include fewer or additional access ports, such that a plurality of access ports 112 are disposed at any location on the port body 140. For example, the surgical evacuation port device 100 can include one access port 112, two access ports 112, three access ports 112, or any other suitable number of access ports 112.

Referring to FIGS. 1-3, each of the access ports 112 includes a valve 116. The valve 116 is coupled to the access port 112 and configured to receive the access tool 302 while maintaining pressure within the primary lumen 142. In the embodiment shown in FIGS. 1-3, two valves 116 are shown coupled to the two access ports 112. The valves 116 may be mounted to the proximal ends of the access ports 112, for example, disposed within the proximal ends of access ports 112. In other embodiments, the valves 116 can be positioned at other suitable location in association with the corresponding access port lumen 118 that enables the access tool 302 to access the primary lumen 142 while maintaining suction pressure within the primary lumen 142. Examples of suitable methods to affix the valves 116 to the corresponding access port include friction fit, press fit, fastening via threads on the inside or outside of the access port lumen 118, via flanges, or seated within. As depicted in FIGS. 1-3, the valves 116 are configured to receive various different types of access tools 302.

Each valve 116 includes a resilient body 180 that defines a passageway 182 that extends longitudinally through the resilient body 180, between a proximal end 181 and a distal end 183. An access tool 302 may be inserted into the passageway 182 by, for example, pushing the access tool into the passageway 182. As shown in FIGS. 1-3, the passageway 182 is configured to deform and accept an access tool 302, such as a sharp or a blunt tool, and maintains a seal around the access tool 302 by closely conforming to the shape of the access tool 302. The access tool 302 may be a syringe 301 or a hollow needle 303, or any other suitable device extending into the access port lumen 118 of the access port 112 and in fluid communication with the primary lumen 142. The resilient body 180 in this embodiment is not punctured by the hollow needle 303 or other sharp point or edge. The resilient body 180 can further receive blunt tools, such as blunt syringe tips without a needle mounted thereon.

The body of the resilient body 180 may include a mating element 188 on the outside surface (i.e., the surface not adjacent the passageway 182) that is configured to interface with at least one complementary mating element 186 provided within the access port lumen 118. For example, in the embodiment of FIG. 2, the body of the resilient body 180 is depicted with mating elements 188 interfacing with complementary mating elements 186 in the access port lumen 118. In the embodiment shown, the complementary mating elements 186 are depicted as shelves and configured to hold the mating elements 188 of the resilient body 180 in a desired location. Other suitable mating elements are within the scope of this disclosure.

The proximal end of the passageway 182 may form a split septum. An access tool inserted into the passageway 182 of the resilient body 180 may deform the split septum to transition the resilient body 180 from the closed position to the open position, while maintaining pressure within the access port lumen 118. The resilient body 180 is shown in a closed state in FIGS. 1-3, the closed state being retained when a tool is not inserted therein. The resilient body 180 is shown closed in FIG. 3. In FIGS. 1-2, the resilient body 180 can be seen in an open state when the access tool 302 is inserted in the passageway 182, thereby providing access to the access port lumen 118. As shown in FIGS. 1-2, in examples where the resilient body 180 includes a split septum, the split septum may be provided at a proximal end 181 of the passageway 182.

In some embodiments, the resilient body 180 is self-sealing. In such embodiments, the resilient body 180 is made from a self-sealing material that is configured to seal punctures, cracks, or damages and to further seal and prevent loss of pressure when the access tool 302 is inserted into and/or removed from the access port 112. Accordingly, when the access tool 302 is inserted, the resilient body 180 can close itself around the access tool, and close completely when the access tool is removed or using adhesive. In other embodiments, the resilient body 180 may be formed from an elastomeric material, for example: silicone, rubber, natural rubber or any other suitable material. In other alternate embodiments, the resilient body 180 can be a collapsible member, such as a sufficiently flexible tube that collapses under a suction applied by the suction device 40 and causes sealing of the access ports 112. In other embodiments, the resilient body 180 is a silicon tip that extends at least partially into the access ports 112. It will be appreciated that the resilient body can be any device suitable for maintaining pressure within the primary lumen 142 while allowing for access to the primary lumen 142.

Depending on the operation, it is sometimes important to maintain the access tool 302 accessing the primary lumen 142 in a single position. To maintain the access tool 302 in place, the access port 112 can include an access port coupling 184 provided on the body of the access port 112. The access port coupling 184 receives corresponding or mating portions from the access tool 302 accessing the surgical evacuation port device 100 to retain the access tool 302 in a desired position and orientation. The access port coupling 184 can be configured to retain a portion of the access tool 302 through the resilient body 180. In the depicted embodiment, the access port coupling 184 includes male luer threads that couple with a female luer of the access tool 302. It will be appreciated that any suitable type of thread can be used and the components with male and female threads can be reversed. In other alternate embodiments, the access port coupling 184 can additionally be any other suitable fitting such as a bayonet fitting, a snap lock, a push to connect fitting or any other suitable fitting appropriate for the application and corresponding access tool 302. Though the access port coupling 184 is depicted as a male locking mechanism, it will be appreciated that it can also be a female locking mechanism or any other suitable mechanism. In other alternate embodiments, the access tool 302 may be used to access the access ports 112 with or without mating with the port fittings. For example, a syringe 301 may access the access port lumen 118 without locking with the access port coupling 184 and may be inserted through the resilient body 180 without being retained in place.

The access tool 302 of the access system 300 may include an access fitting 308 that mates with the access port coupling 184. It will be appreciated that the access fitting 308 is depicted as a luer but may be any fitting that mates with the access port coupling 184. Additionally, although the access fitting 308 is depicted as a female fitting, in other examples it may be a female fitting or any other suitable fitting. As depicted, the access fitting 308 is a luer lock but may be a bayonet, snap to fit, or any other suitable locking mechanism that mates with the corresponding access port coupling 184.

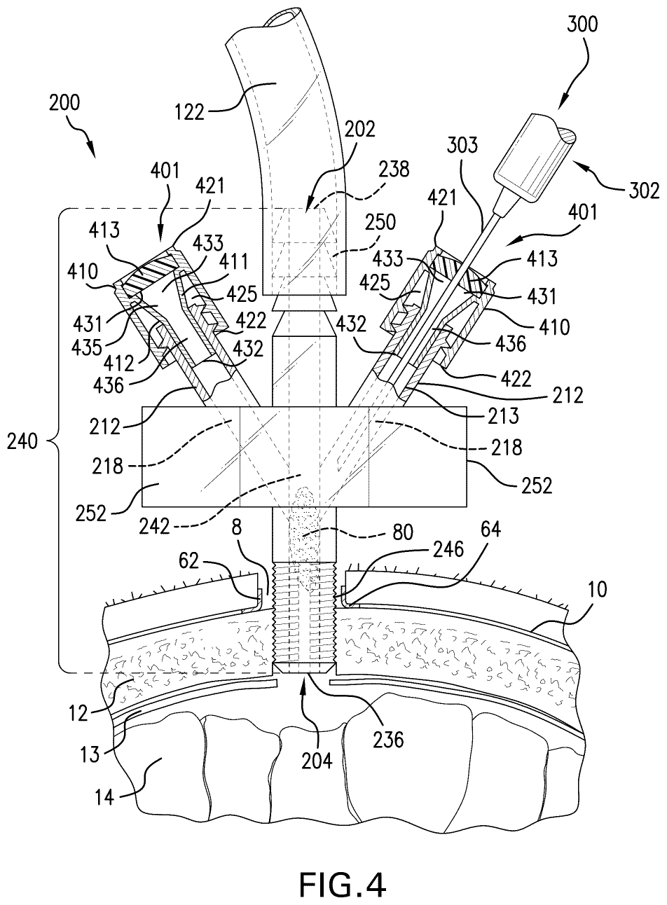

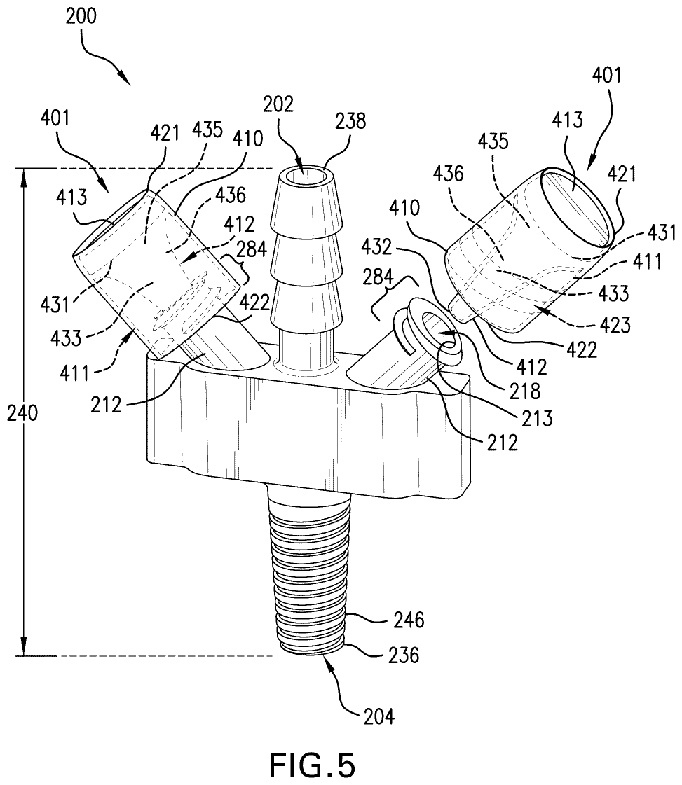

FIGS. 4 and 5 depict another embodiment of a surgical evacuation port device 200. The surgical evacuation port device 200 is configured for use in the surgical evacuation system 20 discussed above. The surgical evacuation port device 200 is connected to a first end of the conduit 122 via a fitting 250. In FIGS. 4 and 5, the fitting 250 is depicted as a barb type fitting. The fitting 250, however, can be any interface that connects the primary lumen 242 to the conduit 122 in an air-tight manner. A second end of the conduit 122 is attached to the suction device 40 in the manner described with respect to FIG. 1. The surgical evacuation port device 200 is an embodiment suitable for at least the enumerated uses discussed above in relation to the surgical evacuation port device 100.

A port body 240 includes the primary lumen 242 that extends through the port body 240 from a distal end 236 to a proximal end 238 and defines a proximal evacuation opening 202 at the proximal end 238 and a distal evacuation opening 204 at the distal end 236. The port body 240 includes at least one access port 212. The access port 212 defines an access port lumen 218 that is in fluid communication with the primary lumen 242. The access port lumen 218 is disposed and oriented with respect to the port body 240 to allow an access tool 302 that is inserted into the access port lumen 218 to optionally extend into the primary lumen 242.

In the embodiment shown in FIGS. 4 and 5, there are two access ports 212 disposed on opposite sides of the port body 240. In some examples, the access ports 212 can be configured as protrusions that extend away from the port body 240. The protrusions (not shown but discussed in the above referenced co-pending U.S. patent application Ser. No. 18/347,474) can be rigid, semi-rigid, or non-rigid protrusions. It will be appreciated that the surgical evacuation port device 200 can also include fewer or additional access ports, such that a plurality of access ports 212 are disposed at any location on the port body 240, as discussed above in relation to the surgical evacuation port device 100.

The access port 212 includes a sealing device 401. The sealing device 401 is coupled to the access port 212 and configured to receive the access tool 302 while maintaining pressure within the primary lumen 142. The sealing device 401 is mounted at a proximal end of the access port 212. In the embodiment shown in FIGS. 4 and 5, two sealing devices 401 are shown respectively mounted to the two access ports 212. For example, as shown in FIG. 4, regarding one of the access ports 212, the sealing device 401 is provided with the access tool 302 inserted, and regarding the other of the access ports 212, the sealing device 401 is provided without the access tool 302 inserted. Except for the sealing devices 401 and mounting thereof, the surgical evacuation port device 200 shown in FIGS. 4 and 5 is substantially similar to the surgical evacuation port device 100 shown and described with reference to FIGS. 1-3.

Referring again to FIGS. 4 and 5, the sealing device 401 can include a resilient member 413, an inner tubular element 411, and a housing 410. The housing 410 may form a substantially cylindrical lumen 425 that extends between a proximal end 421 and a distal end 422 of the sealing device 401. The proximal end 421 includes the resilient member 413. The resilient member 413 can be punctured by the access tool 302, for example, a hollow needle 303. Preferably the resilient member 413 is made from a self-sealing material that is configured to seal punctures, cracks, or damages to the resilient member 413 and prevent loss of pressure when the access tool 302 is inserted into and/or removed from the sealing device 401. Accordingly, when the access tool 302 is inserted, the resilient member 413 can close itself around the access tool 302, and when the access tool 302 is removed, the resilient member 413 can close the puncture hole in order to always maintain the pressure within the access port lumen 218 and primary lumen 242. For example, the seal can be a self-sealing stopper or cover placed within and/or over the proximal end 421, similar to those commonly used for vaccine vials. In other alternate embodiments, the resilient member 413 is a silicone seal that extends at least partially into the cylindrical lumen 425 from the proximal end 421. As another example, the resilient member 413 is a cap that fits over a protrusion of the sealing device 401, where the cap can be held by an adhesive. In other embodiments, the resilient member 413 is a substantially flat seal held in place by a retainer (e.g., a ring fitting over the seal and the side port protrusion.) In other alternate embodiments, the resilient member 413 can be configured as a resilient body 180 having a passageway 182 that extends longitudinally through the resilient body 180.

FIG. 5 illustrates the sealing device 401 coupled to the proximal end of the access port 212 such that the inner tubular element 411 is received within the access port lumen 218 of the access port 212, and the housing 410 is disposed on the outside of the proximal end of the access port 212. At or near the distal end 422 of the sealing device 401, the housing 410 includes one or more coupling elements 423 on an inside wall of the housing 410 that are configured to engage with, or couple with, corresponding or complementary coupling elements provided on the access port 212. The coupling elements 423 can be configured, for example, as threads, luers, latches, etc.

As shown in FIG. 5, the proximal end of the access port 212 includes an access port coupling 284 that is configured to receive, engage, and/or couple with a corresponding coupling element 423 of the sealing device 401. In the depicted embodiment, the access port coupling 284 is configured to include luer threads (male or female) that can couple with the corresponding coupling element 423 that is configured to include luer threads (female or male) of the sealing device 401, which are located on the inside wall of the housing 410. The access port coupling 284 can encompass other fittings, for example, a bayonet fitting, threads, a snap lock, a push to connect fitting or any other suitable fitting for the desired application and corresponding sealing device 401.

As shown in FIGS. 4 and 5, the inner tubular element 411 of the sealing device 401 extends from the inner walls of the housing 410, with respect to a proximal end at or near the resilient member 413, and provides a sealing device lumen 433 for receiving the access tool 302 inserted through the resilient member 413. In this way, the inner tubular element 411 may be configured for guiding the access tool 302 through the sealing device 401. The inner tubular element 411 preferably is arranged concentrically within the housing 410. In addition, the distal end 432 of the inner tubular element 411 can extend beyond the distal end 422 of the housing 410. In other alternate embodiments, the distal end 432 of the inner tubular element 411 is coterminous with the distal end 422 of the housing 410. In still other alternate embodiments, the distal end 432 of the inner tubular element 411 does not extend beyond the distal end 422 of the housing 410.

The inner tubular element 411 is configured to define the sealing device lumen 433 that is disposed within the housing 410 of the sealing device 401. The cross-section of the inner tubular element 411 may be variable or constant between a proximal end 431 and the distal end 432. Accordingly, the sealing device lumen 433 can be configured with a variable or constant cross-sectional area between a proximal end and a distal end. In the embodiment depicted in FIGS. 4 and 5, the inner tubular element 411 is configured to include a first portion 435 and a second portion 436. A proximal diameter of the first portion 435 is configured may be similar to the inner diameter of the housing 410. A distal diameter of the first portion 435 is configured to be smaller than the proximal diameter of the first portion 435. As such, the inner tubular element 411 may be tapered from a proximal end to a distal end of the first portion 435, for example, to form a reducing conical shape. A proximal diameter of the second portion 436 is configured to be substantially similar to the distal diameter of the first portion 435. A distal diameter of the second portion 436 is configured to be smaller than the proximal diameter of the second portion 436 and is coterminous with the distal end 432 of the inner tubular element 411. As shown in the embodiment of FIGS. 4 and 5, the inner tubular element 411 also may be tapered from a proximal end to a distal end of the second portion 436, again forming a reducing conical shape. In other embodiments, the proximal diameter of the second portion 436 can be substantially similar to the distal diameter of the second portion 436. In this instance, the diameter of the inner tubular element may be substantially uniform, through the second portion 436. The inner tubular element 411 may have a conically reducing profile through the first portion 435 and a cylindrical profile through the second portion. In other alternate embodiments, the cross-sections of the first portion 435 and the second portion 436 are not circular.

The inner tubular element 411 includes an outer surface 412. The outer surface 412 of at least a portion of the inner tubular element 411 can be configured to form a seal with an inner surface 213 of the access port 212. As shown in FIGS. 4 and 5, the second portion 436 of the inner tubular element 411 is configured with a gradual reducing taper between the proximal end the distal end. For clarity, in FIG. 5, the taper of the second portion 436 of the inner tubular element 411 has been exaggerated. The taper can be configured such that once a suitable length of the inner tubular element 411 is inserted into the access port lumen 218 of the access port 212, the outer surface 412 forms an interference fit with the inner surface 213 of the access port 212. The interference between outer surface 412 of the inner tubular element 411 and the inner surface 213 of the access port 212 is configured to form the seal.

In other alternate embodiments, the outer surface 412 of the first portion 435 of the inner tubular element 411 is configured to form the seal. For example, the second portion 436 of the inner tubular element 411 can be configured to be inserted into the access port lumen 218 of the access port 212 without substantial interference. The taper of the first portion 435 can be configured such that once a suitable length of the inner tubular element 411 is inserted into the access port lumen 218 of the access port 212, the outer surface 412 of the first portion 435 forms the interference fit with the inner surface 213 of the access port 212. In other examples, the seal between the access port 212 and the sealing device 401 can be formed by outer surface 412 of the second portion 436, the outer surface 412 of the first portion 435, or both.

As discussed above, the access tool 302 can be inserted into the sealing device 401, which is mounted on the access port 212, by puncturing the resilient member 413 (e.g., using the hollow needle 303), and therefore gaining access into the access port lumen 218 via the inner tubular element 411. Pressure is maintained within the primary lumen 242 because the resilient member 413 maintains a seal around the access tool 302 by closely conforming to the shape of the tool, and at least a portion of the inner tubular element 411 forms a seal with the inner walls of the access port lumen 218.

The surgical evacuation port device 200 includes a bone engagement portion 246. The bone engagement portion 246 is configured to mount the port body 240 through a hole 8 in a bone (e.g. the skull 12) to aspirate fluid from the bone and/or surrounding areas thereof, through the primary lumen 242 when a suction pressure is applied. Other aspects of the bone engagement portion 246 are similar to those discussed above in relation to the surgical evacuation port device 100.

Use of the access tool 302 with the surgical evacuation port device 100 (see FIGS. 1-3) or the surgical evacuation port device 200 (see FIGS. 4 and 5) is described in greater detail herein. As described above, the access system 300 can include a syringe 301 (see FIGS. 1-3). The syringe 301 may be attached to the hollow needle 303, a cannula (not shown), or other device for assistance with removing the blockage 80 or other operation. The syringe 301 includes a distal end 304 that is hollow and extends through either the passageway 182 of the resilient body 180 of the valve 116 in the surgical evacuation port device 100 or punctures the resilient member 413 and extends through the inner tubular element 411 of the sealing device 401 of the surgical evacuation port device 200. As depicted in FIG. 4, for example, the distal end 304 comes to a tip which is narrower than the rest of the body. The syringe 301 can be of any suitable type and the distal end 304 can be configured in various ways. For example, the distal end can be configured as a luer lock, luer slip, or any others.

Referring to FIGS. 1-3, the syringe includes a barrel 310 that has a volume. The syringe 301 can include a plunger (not shown). The plunger can be airtight within the barrel 310. When pushed or pulled, the plunger causes negative or positive pressure to build within the barrel of the syringe 301. The negative pressure can assist with removing the blockages 80. Positive pressure can be used to inject medication such as thrombolytics. The access system 300 may be any other system or device for accessing the primary lumen 142, 242 without interrupting the process of fluid evacuation.

Additionally, or alternatively, the barrel 310 of the syringe 301 can carry medication (i.e., a thrombolytic agent, heparin, or any other medication) which can be injected through the access port into the primary lumen 142, 242. For example, the barrel 310 can be filled with a thrombolytic agent which can be introduced to the blockage by depressing the plunger in the distal direction for delivering the thrombolytic agent from the syringe barrel 310 at, near, or into the blockage 80. The thrombolytic agent may at least partially dissolve the blockage. The syringe 301 may then, optionally, be used to suction the debris of the thrombolized blockage and/or any non-thrombolized portions of the blockage by, for example, withdrawing, the plunger proximally towards a user to create the suction force. Optionally, the debris of the thrombolized blockage and/or any non-thrombolized portions of the blockage may be aspirated via the primary lumen 142, 242 along with the fluid being evacuated.

Some studies have shown that after draining or during drainage of the subdural hematoma, continuous irrigation or post-surgery irrigation reduces future risk of hematomas. As such, a system (not shown) for irrigating the subdural space can be fitted to the access ports. For example, the syringe 301 or other device can provide fluid such as sterile saline to the first access port 112, 212 while a suction system removes the fluid through a second access port 112, 212. It will be appreciated that the above examples are exemplary, and any other tools or combination of tools may be used with the access ports 112, 212 to assist with withdrawal, healing, cleaning, or any other suitable usage where access to the evacuation port while maintaining suction through the primary lumen 142, 242 is required.

The surgical evacuation port device 100, 200 may be configured to be substantially or entirely optically transparent. As discussed above, identification and diagnosis allowing for removal of blockages 80 is important. The viewing portion may additionally be useful during irrigation, aspiration, or any other operation allowing the operator to maintain view of what is occurring within the surgical evacuation port device 100, 200. The access ports 112, 212 and the valve 116 or sealing device 401 in combination with the rest of the surgical evacuation port device 100, 200 being transparent, may allow the user to detect the blockage 80; determine the location and properties of the blockage 80; monitor the location and/or passage of the access tool 302 through the access ports 112, 212 into the access port lumens 118, 218 and further into the primary lumen 142, 242 or even extending from distal evacuation opening 104, 204; determine the effectiveness of a thrombolytic agent, if the access tool 302 is used to inject a thrombolytic agent; determine the effectiveness of suctioning the blockage 80 with the access tool 302; and/or determine if the blockage 80 requires further intervention such as injection of more thrombolytic agent, change in location of the hollow needle 303, withdrawal/suction via the hollow needle 303, attachment of a different device or any other suitable change. The user may also optically visualize the blockage 80 (and/or pieces or debris thereof) if it is suctioned out via the access port lumen 118, 218 before, during, and/or after performance of the operation for removal of the blockage 80.

In other alternate embodiments, the surgical evacuation port device 100, 200, can include one or more viewing portions. The viewing portion can be a portion or an entire component of the surgical evacuation port device 100, 200 that is optically transparent, for example, the primary lumen 142, 242, port body 140, 240, access port lumens 118, 218, bone engagement portion, 146, 246, valve 116 or sealing device 401, etc. The surgical evacuation port device 100, 200 and/or any of its individual components can be configured as a viewing portion.

In other alternate embodiments, the viewing portion may be an optically transparent window provided in one or more components, for example, the primary lumen 142, 242, the port body 140, 240, the access port lumens 118, 218, the bone engagement portion, 146, 246, the valve 116 or sealing device 401, etc. In alternate embodiments, other portions of the surgical evacuation port device 100, 200 apart from the viewing portion are less transparent compared to the viewing portion, non-transparent, or opaque.

As used herein, optically transparent refers to light being allowed to pass through the material with minimal absorption. In some alternate embodiments, the optically transparent material has a high transparency meaning it allows at least a threshold percentage of light to pass through (e.g., greater than 90%, greater than 95%, or any other suitable percentage) such that it is essentially optically see-through. In some alternate embodiments, the optically transparent material has a medium or medium low transparency or any other suitable transparency allowing at least a threshold of light to pass through (e.g., about greater than 60%, greater than 75%, or any other suitable percentage) that is enough to allow the user to detect a blockage and determine the cause of a blockage, its properties (e.g., size, shape, density, etc.), and/or its location.

For example, a user may visually or optically observe the interior of the primary lumen 142, 242 to detect that the blockage 80 has occurred via the viewing portion. Furthermore, the user may view the blockage 80 via the viewing portion to determine the cause of the blockage 80 as being a clot, its properties (e.g., size, shape, density, etc.), and/or its location. The user may use the information to determine an appropriate corrective action for removal of the blockage 80 (for example, by injection of a thrombolytic agent, by breakage, by repositioning, and/or by suction).

In some examples, the entire surgical evacuation port device 100 200 is formed from an optically transparent material. In some examples, certain components or portions of the surgical evacuation port device 100 are entirely formed from an optically transparent material. The viewing portion therefore surrounds such components of the surgical evacuation port device 100, 200. For example, the port body 140, 240 of the surgical evacuation port device 100, 200 can be optically transparent such that the viewing portion surrounds the primary lumen 142, 242 within the port body 140, 240 allowing a user to see within the interior of the primary lumen 142, 242. This may allow a surgeon or user to see within the primary lumen 142, 242 and optically detect the blockage 80 within the primary lumen 142, 242. In some other examples, only a portion of the port body 140, 240 that defines the primary lumen 142, 242 (and/or the primary lumen 142, 242 itself) are the viewing portion and are made from an optically transparent material that allows a user to optically see into the primary lumen 142, 242 to view obstructions. Typically, the viewing portion of the port body 140, 240 that would be transparent is a location where the blockages 80 commonly form, such as near the distal end 136, 236 of the primary lumen 142, 242.

In some embodiments, the bone engagement portion 146, 246 of the port body is optically transparent and forms the viewing portion. In some other examples, the viewing portion may be a window formed from an optically transparent material located on the surgical evacuation port device 100, 200. For example, a window may be formed on the port body 140, 240 to allow the user to see within the primary lumen of the device and determine whether a blockage has occurred. Typically, the window may be formed on the port body at a location where the blockages 80 commonly form such as near the distal end of the primary lumen 142, 242.

Optionally, the user may optically visualize the blockage 80 without the use of non-optical imaging technologies (optical imaging technologies such as a microscope may be used). Optionally, the user may also optically visualize the blockage 80 (and/or pieces or debris thereof) if it is suctioned out via the primary lumen 142, 242 before, during, and/or after performance of the operation for blockage removal.

In addition to the viewing portions or windows discussed above for detection of the blockage 80 and/or determining its location and properties, viewing portions may be provided and/or configured such that a user can determine the location and/or monitor the progress of the access tool 302, for example, the distal end 304 of the syringe 301, the hollow needle 303, an irrigation device or any other device, within the access port lumens 118, 218 and/or the primary lumen 142, 242 during an operation for removal of the blockage 80.

For example, as depicted in FIGS. 1, 2, and 5, at least a portion of the port body 140, 240 that surrounds the access ports 112, 212 along with certain portions of the access ports 112, 212 may be optically transparent. The transparency can allow a surgeon to optically visualize and monitor an opaque or semitransparent needle or access tool that is inserted into the access port, determine its location at any time, and/or assess effectiveness of the operation performed by the access tool 302.

In some alternate embodiments, the viewing portion can be a window made of optically transparent material that is located on the surgical evacuation port device 100, 200 (e.g., glass, a transparent polymer, or any other suitable transparent material) such that a user can see within the access port lumen and determine the location of the tool or needle.

In other alternate embodiments, only the viewing portion of the surgical evacuation port device 100, 200, such as the window or portion of the port body, can be optically transparent to allow the user to see within the device and detect a blockage and determine the location of an access tool relative to the blockage while the rest of the device is less transparent or opaque.

In some embodiments, the access ports 112, 212, the access port lumens 118, 218, and the primary lumen 142, 242 all include (or are surrounded by) one or more strategically located viewing portions or windows (and/or are fully transparent) to allow the user to see within the primary lumen/access lumens and monitor an access tool such as a needle as it extends through the access lumen and into the primary lumen while the rest of the surgical evacuation port device 100, 200 is opaque or less transparent.

In some embodiments, the port body 140, 240 the access ports 112, 212 or the viewing portion of the surgical evacuation port device 100, 200 is formed from an optically transparent or an optically semi-transparent (as discussed above) material. In some embodiments, the material is a polymer. In other alternate embodiments, the material can include, without limitation, crystalized ABS, polycarbonate, or a transparent ceramic such as aluminum oxynitride, glass, acrylic or any other medical grade, shatter resistant materials. In other embodiments, the surgical evacuation port device 100, 200 can be formed from a material that has a substantially low thrombogenicity. In other embodiments, the surgical evacuation port device 100, 200 can be coated with a material having low thrombogenicity. In other embodiments the surgical evacuation port device 100, 200 can be formed from a material that has a substantially low thrombogenicity, is optically transparent, and is sufficiently rigid to sustain a vacuum. Any combination of properties discussed above are also possible.

The port body 140, 240, or other portions of the surgical evacuation port device 100, 200 can be made of suitably rigid materials, stiff plastics, glass, transparent ceramic or any other suitable material. In some embodiments, the surgical evacuation port device 100, 200 is made from a material that is sufficiently rigid to be mounted into bone (e.g. the skull 12 of a human). In some examples, the surgical evacuation port device 100, 200 is formed from a material that is sufficiently rigid to not collapse when subjected to suction pressure applied for removal of fluid and or the blockage. In some embodiments, the material is sufficiently stiff polymer. In other alternate embodiments, the surgical evacuation port device 100, 200 can be made from a material capable of withstanding common sterilization techniques and generally suitable for surgical instruments, for example, surgical grade stainless steel, titanium, tungsten carbide, Polyetheretherketone (PEEK) and nitinol.

In some embodiments, the surgical evacuation port device 100, 200 is made from and/or coated a material having a substantially low thrombogenicity (e.g., thrombogenicity refers to the tendency to of material in contact with blood to form a thrombus or clot), such that it does not cause blood clotting or minimizes blood clotting. In some embodiments, the material is substantially less thrombogenic than stainless steel. In some examples, the material is half as thrombogenic as stainless steel. In other examples, the material is less than half as thrombogenic as stainless steel. Other suitable embodiments are additionally possible. In some embodiments, the portion that engages with the skull 12 (e.g., the bone engagement portion 146, 246) is formed from a material having a material having a substantially low thrombogenicity. Examples of the thrombogenic material include, without limitation, polypropylene, PET, etc.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

What is claimed is:

1. A surgical evacuation port device, comprising:
a body that includes:
a distal opening,
a primary evacuation opening, and
a primary lumen extending from the distal opening to the primary evacuation opening;
an access port defining an access port lumen that is in fluid communication with the primary lumen; and
a sealing device mounted at a proximal end of the access port and configured to be penetrated by an access tool while maintaining pressure within the primary lumen, the sealing device comprising:
a housing configured for mounting the sealing device at the proximal end of the access port;
an inner tubular element having an outer surface, wherein the outer surface of the inner tubular element is configured to form a seal with the access port; and
a sealing device lumen defined by the inner tubular element and disposed within the housing.

2. The surgical evacuation port device of claim 1, wherein the inner tubular element is arranged concentrically within the housing.

3. The surgical evacuation port device of claim 1, wherein the outer surface of the inner tubular element forms an interference fit with an inner surface of the access port, the interference fit configured to form the seal with the access port.

4. The surgical evacuation port device of claim 3, wherein the outer surface of the inner tubular element is wider at a proximal end than at a distal end to form a taper, the distal end configured to fit within the access port.

5. The surgical evacuation port device of claim 1, wherein the inner tubular element is configured for guiding the access tool through the sealing device.

6. The surgical evacuation port device of claim 1, wherein the inner tubular element extends distally from an inner wall of the housing and defines a lumen for receiving the access tool.

7. The surgical evacuation port device of claim 1, wherein the sealing device further comprises a resilient member configured to be penetrated by the access tool.

8. The surgical evacuation port device of claim 7, wherein the resilient member is arranged proximally from the inner tubular element.

9. The surgical evacuation port device of claim 7, wherein the resilient member is configured as a resilient body having a passageway that extends longitudinally through the resilient body.

10. The surgical evacuation port device of claim 7, wherein the resilient member is made from a self-sealing material configured to seal punctures, cracks, or damage.

11. The surgical evacuation port device of claim 1, wherein the housing comprises at least one coupling element on an inside wall configured to engage with a corresponding coupling element on the access port.

12. The surgical evacuation port device of claim 1, wherein the body is made from a rigid material with a substantially low thrombogenicity.

13. The surgical evacuation port device of claim 1, wherein the device is optically transparent.

14. The surgical evacuation port device of claim 1, wherein the access tool is a syringe.

15. The surgical evacuation port device of claim 1, wherein the syringe comprises a hollow needle.

16. The surgical evacuation port device of claim 1, further comprising a valve comprising a resilient body having a passageway that extends longitudinally through the resilient body, the valve being coupled to the sealing device.

17. A surgical evacuation system, comprising:
the surgical evacuation port device of claim 1;
a suction device configured to provide a suction force through the primary lumen; and
the access tool comprising at least one syringe.

18. The surgical evacuation system of claim 16, wherein the syringe is operable with the surgical evacuation port device to cause negative pressure to assist with removing blockages or cause positive pressure for injecting medication.

19. A surgical evacuation port device, comprising:
a body that includes:
a distal opening,
a primary evacuation opening, and
a primary lumen extending from the distal opening to the primary evacuation opening;
an access port defining an access port lumen that is in fluid communication with the primary lumen; and
a valve coupled to the access port and configured to receive an access tool while maintaining pressure within the primary lumen, the valve comprising a resilient body having a passageway that extends longitudinally through the resilient body.

20. The surgical evacuation port device of claim 19, wherein the passageway forms a split septum provided at a proximal end of the resilient body.

21. A surgical evacuation system, comprising:
the surgical evacuation port device of claim 19;
a suction device configured to provide a suction force through the primary lumen; and
the access tool comprising at least one syringe.

22. The surgical evacuation system of claim 21, wherein the syringe is operable with the surgical evacuation port device to cause negative pressure to assist with removing blockages or cause positive pressure for injecting medication.

23. A method for removing subdural fluids, comprising:
drilling a hole through a skull and a dura of a patient;
mounting a surgical evacuation port device to the hole, the surgical evacuation port device comprising:
a body that includes:
a distal opening,
a primary evacuation opening, and
a primary lumen extending from the distal opening to the primary evacuation opening;
an access port defining an access port lumen that is in fluid communication with the primary lumen; and
a sealing device coupled to the access port and configured to be penetrated by an access tool while maintaining pressure within the primary lumen, the sealing device comprising:
a housing configured for mounting the sealing device at the proximal end of the access port;
an inner tubular element having an outer surface, wherein the outer surface of the inner tubular element is configured to form a seal with the access port; and
a sealing device lumen defined by the inner tubular element and disposed within the housing;
using a suction device configured to provide a suction force through a primary lumen of the surgical evacuation port device;
evacuating subdural fluid from the dura through the primary lumen;
inserting the access tool through the access port; and
assisting with withdrawal or cleaning when access is required while the suction force is maintained in the primary lumen.

24. The method of claim 23, further comprising inserting a hollow needle through the access port lumen into the primary lumen.

25. The method of claim 24, further comprising injecting a thrombolytic agent via the hollow needle.

26. The method of claim 24, further comprising suctioning a blockage via the hollow needle.

27. The method of claim 23, further comprising irrigating the subdural space by administering sterile fluid through a first access port and evacuating said fluid through a second access port.
